(19) United States
(12) Patent Application Publication
(10) Pub. No.: US 2003/0217372 A1
(43) Pub. Date: Nov. 20, 2003

(54) Methods of using alpha 1b-adrenergic receptors

(76) Inventor: Susanna Cotecchia, Lausanne (CH)

Correspondence Address: Ivor R. Elrifi, Mintz, Levin, Cohn, Ferris,, Glovsky and Popeo, P.C., One Financial Center, Boston, MA, 02111, US

(21) Appl. No.: 10/396,952

(22) Filed: Mar. 25, 2003

Related U.S. Application Data
(60) Provisional application No. 60/367,833, filed on Mar. 25, 2002.
(60) Provisional application No. 60/394,423, filed on Jul. 8, 2002.

(51) Int. Cl. A61K 31/00; A01K 67/00

(52) U.S. Cl. 800/3; 800/8; 514/1

(57) ABSTRACT

The present invention relates generally to α-1b-adrenergic receptors and to methods for use of α1b-ARs. In particular, the invention relates to the use of such methods for the identification of modulators of α1b-adrenergic receptor activity.

### RELATED APPLICATIONS

This application claims the benefit of U.S. S. No. 60/367,833, filed Mar. 25, 2002, and U.S. S. No. 60/394,423, filed Jul. 8, 2002, both of which are incorporated herein by reference in their entireties.

### FIELD OF THE INVENTION

The present invention relates generally to alpha-1b-adrenergic receptors (α-1b-adrenergic receptors or α1b-ARs) and to methods for the use alpha-1b-adrenergic receptors including methods for the identification of modulators of alpha-1b-adrenergic receptor activity.

### BACKGROUND OF THE INVENTION

Adrenergic receptors are integral membrane proteins which have been classified into two broad classes, the alpha and the beta adrenergic receptors. Both types of receptors mediate the action of the peripheral sympathetic nervous system upon binding of catecholamines, including norepinephrine and epinephrine.

Norepinephrine is produced by adrenergic nerve endings, while epinephrine is produced by the adrenal medulla. The binding affinity of adrenergic receptors for these compounds forms one basis of the classification: alpha receptors bind to norepinephrine more strongly than epinephrine and much more strongly than the synthetic compound isoproterenol. The binding affinity of these hormones is reversed for the beta receptors. In many tissues, the functional responses, such as smooth muscle contraction, induced by alpha receptor activation are opposed to responses induced by beta receptor binding.

The functional distinction between alpha and beta receptors was further highlighted and refined by the pharmacological characterization of these receptors from various animal and tissue sources. As a result, alpha and beta adrenergic receptors have been further subdivided into α1, α2, β1 and β2 subtypes. Functional differences between α1 and α2 receptors have been recognized, and compounds which exhibit selective binding between these two subtypes have been developed. For a general background on the α-adrenergic receptors, see Robert R. Ruffolo, Jr., α-Adrenoreceptors: Molecular Biology, Biochemistry and Pharmacology, (Progress in Basic and Clinical Pharmacology series, Karger, 1991).

### SUMMARY OF THE INVENTION

In one aspect, the present invention involves methods for identifying a modulator of alpha1b-adrenergic receptor activity, including the steps of providing a first mammal having a modified alpha1b-adrenergic receptor gene, administering to the first mammal a candidate agent, measuring a detectable phenotype of the first mammal, and comparing the detectable phenotype of the first mammal with a detectable phenotype of a second mammal. An alteration in the detectable phenotype of the first mammal compared to the second mammal indicates that the candidate agent is a modulator of alpha1b-adrenergic receptor activity. In some embodiments, the detectable phenotype is an increase in an alpha1b-adrenergic receptor activity, a decrease in an alpha1b-adrenergic receptor activity, a restoration of an alpha1b-adrenergic receptor activity such that the activity is similar to the activity of a wild-type alpha1b-adrenergic receptor, locomotor response, the level of extracellular dopamine in the nucleus accumbens, addiction to one or more addictive compounds, or conditioned place preference. The present invention also relates to the modulators identified by these methods.

In a second aspect, the present invention involves methods for identifying a modulator of alpha1b-adrenergic receptor activity, including the steps of administering to a test mammalian cell containing a modified alpha1b-adrenergic receptor gene a candidate agent, measuring a detectable response by the test mammalian cell, and comparing the detectable response of the test mammalian cell with a reference response. A change in the detectable response of the test mammalian cell relative to the reference response indicates that the candidate agent is a modulator of alpha1b-adrenergic receptor activity. In various embodiments of the present invention, the detectable response includes a change in the level of dopamine secreted by the test mammalian cell. The mammalian cell may be obtained from a rodent, such as a rat or a mouse. The present invention also relates to the modulators identified by these methods.

In a third aspect, the present invention involves methods of identifying a compound that inhibits or reduces drug addiction. In these methods, a first mammal is provided, where the first mammal contains a modified alpha1b-adrenergic receptor gene, where the modified alpha1b-adrenergic receptor gene is a non-functional alpha1b-adrenergic receptor gene or an alpha1b-adrenergic receptor gene with reduced function as compared to a wild-type alpha1b-adrenergic receptor gene. A candidate agent is administered to the first mammal and a detectable behavior of the first mammal is measured, where the detectable behavior is correlated with an addiction. Finally, the detectable behavior of the first mammal is compared with a detectable behavior of a second mammal, where the second mammal does not have a modified alpha1b-adrenergic receptor gene. A decrease in the detectable behavior of the second mammal relative to the first mammal indicates that the candidate agent is a compound that inhibits or reduces addiction. The present invention also relates to compounds identified by this method.

In a fourth aspect, the present invention involves methods of identifying a compound that promotes addiction. A first mammal containing a modified alpha1b-adrenergic receptor gene is provided, where the modified alpha1b-adrenergic receptor gene is a non-functional alpha1b-adrenergic receptor gene or an alpha1b-adrenergic receptor gene with reduced function as compared to a wild-type alpha1b-adrenergic receptor gene. A candidate agent is administered to the first mammal and a detectable behavior of the first mammal is measured, where the detectable behavior is correlated with an addiction. Finally, the detectable behavior of the first mammal is compared with a detectable behavior of a second mammal, which does not have a modified alpha1b-adrenergic receptor gene. An increase in the detectable behavior of the second mammal relative to the first mammal indicates that the candidate agent is a compound that promotes addiction. The present invention also relates to compounds identified by this method.

In a fifth aspect, the present invention involves methods of identifying an inducer of an alpha1b-adrenergic receptor-associated disorder by providing a first mammal containing a modified alpha1b-adrenergic receptor gene, administering a candidate agent to the first mammal, measuring a detectable phenotype of the first mammal, and comparing the detectable behavior of the first mammal with a detectable behavior of a second mammal, where the second mammal does not have a modified alpha1b-adrenergic receptor gene. An increase in the detectable behavior of the second mammal relative to the first mammal indicates that the candidate agent is an inducer of an alpha1b-adrenergic receptor-associated disorder. The present invention also relates to the inducers identified by this method.

In a sixth aspect, the present invention involves methods of identifying a repressor of an alpha1b-adrenergic receptor-associated disorder, by providing to a first mammal containing a modified alpha1b-adrenergic receptor gene, administering a candidate agent to the first mammal, measuring a detectable phenotype of the first mammal, and comparing the detectable behavior of the first mammal with a detectable behavior of a second mammal that does not have a modified alpha1b-adrenergic receptor gene. A decrease in the detectable behavior of the second mammal relative to the first mammal indicates that the candidate agent is a repressor of an alpha1b-adrenergic receptor-associated disorder. The present invention also relates to repressors identified by this method.

In some embodiments, the modified alpha1b-adrenergic receptor gene is a non-functional alpha1b-adrenergic receptor gene. In alternative embodiments, the modified alpha1b-adrenergic receptor gene is an alpha1b-adrenergic receptor gene having reduced function as compared to a wild-type alpha1b-adrenergic receptor gene.

In certain embodiments, the second mammal does not have a modified alpha1b-adrenergic receptor gene. In certain embodments, the candidate agent is an agonist, a partial agonist, an inverse agonist, a partial inverse agonist, and an antagonist. For example, the candidate agent may be an alpha1b-adrenergic receptor agonist including norepinephrine, oxymetazoline, methoxamine, phenylephrine, dopamine, and similar compounds, an alpha1b-adrenergic receptor inverse agonist including quinazolines (e.g., prazosin), N-arylpiperazines, and similar compounds, or an alpha1b-adrenergic receptor antagonist including labetolol, phentolamine, phenoxybenzamine, and similar compounds. The candidate agent may act as an agonist and an antagonist.

In other embodiments, the route of administration can be oral administration or parenteral administration. Parenteral administration includes, for example, subcutaneous administration, subdermal administration, intraarterial administration, intravenous administration, intraperitoneal administration, topical administration, intramuscular administration, ophthalmic administration, nasal administration, and otic administration. Administration of a candidate agent or other compound to a mammalian cell includes direct injection of the agent or compound (e.g., by microinjection), by contacting the agent or compound with the cell culture media contacting the mammalian cell, or treating a surface or other material with an agent and contacting the agent-treated surface with the mammalian cell.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and are not intended to be limiting.

Other features and advantages of the invention will be apparent from the following detailed description and from the claims.

### BRIEF DESCRIPTION OF THE DRAWINGS

FIG. 1 is a series of autoradiographic images in sectioned WT and α1b-AR KO mice brain that are quantified in histograms, which demonstrates the localization of catecholamine transmission markers in WT and α1b-AR KO mice. FIG. 1A is an autoradiographic image showing the localization of al-adrenergic receptors revealed by 3H-prazosin (1 nM). Binding densities were quantified in the cortex (layer III) and the thalamus (N=4 animals per group). FIG. 1B is an autoradiographic image showing the localization of DI and D2 dopamine receptors (DiR and D2R), dopamine transporter (DAT) and vesicular monoamine transporter (VMAT) revealed respectively by 3H-SCH23390,1251-iodosulpride, 3H-W1N35,428 and 3H-tetrabenazine. FIG. 1C is a histogram that depicts the binding densities as determined in FIG. 1A in the striatum (N=4 animals per group). FIG. 1D is a histogram demonstrating the formation of cyclic AMP in striatal membranes under basal conditions or in response to DA (100 μM) (N=4 animals per group).

FIG. 2 is a series of dose response graphs that depicts the results of locomotor response to novelty, saline, scopolamine and chloro-APB experiments in WT and α1b-AR KO mice. FIG. 2A shows the locomotor response to novelty as measured every 5 minutes during the first 50 minutes mice spent in the experimental apparatus. FIG. 2B shows the the locomotor response to saline and FIG. 2C shows the the locomotor response to scopolamine. Animals were placed in the experimental apparatus for 90 minutes, received a saline injection and were replaced in the apparatus for 60 minutes. On the following day, animals were placed in the experimental apparatus for 90 minutes, they received an intraperitoneal injection of either saline or scopolamine (1 mg/kg) and their locomotor response was measured every 5 minutes for 60 minutes. FIG. 2D represents the results of locomotor response to chloro-APB; animals were placed in the experimental apparatus for 90 minutes, received a saline injection and were replaced in the apparatus for 60 minutes. On the following days, animals were placed in the experimental apparatus for 90 minutes, they received an intraperitoneal injection of chioro-APB and their locomotor response was measured every 5 minutes for 60 minutes. Several doses of chloro-APB were tested on consecutive days in a random order. Groups of 8 to 14 animals were used in all these experiments shown in FIGS. 2A-D.

FIG. 3 is a series of histograms and time course graphs that depict the locomotor responses to D-amphetamine, cocaine and morphine in WT and α1b-AR KO mice. Locomotor responses to different doses of D-amphetamine, cocaine and morphine were measured every 5 minutes under conditions similar to that in FIG. 2B except that locomotor response to morphine was measured for 120 minutes. Independent groups of animals were used for each treatment to avoid eventual behavioral sensitization (N=6-16 per group). FIG. 3A demonstrates the total locomotor responses as measured during the first hour following the drug administration are presented in function of the dose. *p<0.05 and **p<0.01 when WT and α1b-AR KO mice locomotor responses were significantly different from basal locomotor responses. °p<0.05 and °°p<0.01 when WT and α1b-AR KO mice locomotor responses were significantly different (Student's t-test). FIG. 3B depicts time course analyses of locomotor responses measured every 5 minutes are illustrated for D-amphetamine (2 mg/kg), cocaine (15 mg/kg) and morphine (7.5 mg/kg).

FIG. 4 is a series of histograms and time course graphs that shows the prazosin effect on the locomotor responses of WT and α1b-AR KO mice to D-amphetamine, cocaine and morphine. Animals were placed in the experimental apparatus for 150 minutes, received two saline injections after 60 minutes and 90 minutes spent in the corridor. On the following day, animals were placed in the experimental apparatus for 60 minutes, they received an intrapenitoneal injection of either saline or prazosin (1 mg/kg) and were replaced in the corridor for 30 minutes. Then, they received an intraperitoneal injection of D-amphetamine, cocaine or morphine and their locomotor responses was measured every 5-min for 60 minutes or 120 minutes. Independent groups of animals were used for each treatment to avoid eventual behavioral sensitization (N=6-12 per group). Locomotor responses measured during the first hour following the injection are presented in histograms in FIG. 4A and time courses are illustrated in FIG. 4B. *p<0.05 and **p<0.01 when locomotor responses after prazosin pre-treatment were significantly different from locomotor responses after saline pre-treatment (Student's t-test). °p<0.05 significantly different from WT mice locomotor responses (Student's t-test).

FIG. 5 is a series of dose response graphs that demonstrates the induction of locomotor sensitizations induced by repealed administration of D-amphetamine, cocaine and morphine in WT and α1b-AR KO mice. Animals spent 90 minutes in the experimental apparatus, received a saline injection and were replaced in the apparatus for 60 minutes. On the following day, they spent 90 minutes in the experimental apparatus, received an intraperitoneal injection of saline, morphine, cocaine or D-amphetamine and their locomotor response was measured for 60 minutes or 120 minutes. Four similar sessions took place every other day. The sixth session took place after a 10-day withdrawal. Locomotor responses measured during the first hour following each injection are presented in function of the number of injections and slope values. N=6 to 15 animals per group.

FIG. 6 is a series of histograms and time course graphs that depicts the expression of locomotor sensitizations induced by repeated administration of D-amphetamine, cocaine and morphine in WT and α1b-AR KO mice. Locomotor responses to D-amphetamine, cocaine and morphine were measured in naive mice and in mice having previously received 5 drug injections as described in FIG. 5. N=6 to 12 animals per group. FIG. 6A shows the total locomotor responses measured during the first hour following the drug injection. *p<0.05, **p<0.01 and ***p<0.001 significantly different between WT and α1b-AR KO mice. °p<0.05, °°p<0.01 and °°°p<0.00i significantly different from respective naive mice. FIG. 6B represents the time course of the locomotor responses measured every 5 minutes.

FIG. 7 is a series of histograms and dose response graphs that compares the oral consumption of cocaine, morphine, sucrose and quinine in a two-bottle choke paradigm in WT and α1b-AR KO mice. The consumption of water, cocaine (0.2 mg/ml), morphine (0.15 mg/ml) and different concentrations of sucrose and quinine were measured in a two-bottle choice paradigm, and were expressed in % of total fluid intake. N=7 to 9 animals per group. *p<0.05, **p<0.01 when cocaine or morphine consumption significantly differed from water consumption (paired Student's t-test). °p<0.05, °°°p<0.001 when consumption of α1b-AR KO mice significantly differed from consumption of WT mice (unpaired Student's t-test).

FIG. 8 is a series of histograms and dose response graphs that represents the results of a conditioned place preference experiment induced by morphine in WT and α1b-AR KO mice. WT and α1b-AR KO mice were conditioned to receive morphine (5 mg/kg s.c.) in a compartment and a saline injection in the other compartment. The saline group received saline injections in both compartments. N=6 to 11 animals per group. The upper left graph show the time spent in the morphine-associated compartment before conditioning (pre-test), after four morphine injections and four saline injections (post-test 1) and after two supplementary morphine and two supplementary saline injections (post-test 2). *p<0.05 and **p<0.01 when time spent in the drug compartment was different between pretest and post-test 1 or 2 (paired Student's t-test). The upper right graph show scores that correspond to the time spent in the morphine compartment during the post-test (1 and 2) minus time spent in the morphine compartment during the pre-test. p<0.05 when scores were higher for morphine treated mice than for saline treated mice (unpaired Student's t-test). The lower left and lower right graphs show the locomotor activity measured during the 30 minutes of each of the 12 conditioning sessions corresponding either to morphine or to saline injection. ***p<0.001 locomotor activity measured during the morphine session was higher than during the corresponding saline session (paired Student's t-test).

FIG. 9 is a series of images of sectioned mouse and rat brain that demonstrates the localization of dialysis probes in the nucleus accumbens. Mouse (top) and rat slices (bottom) (100 μm thick) were stained with safranine.

FIG. 10 is a series of histograms and time course graphs that demonstrates the effects of systemic D-amphetamine on extracellular DA levels in the nucleus accumbens and locomotor activity in WT and α1bAR-KO mice. D-amphetamine was injected 240 minutes after the introduction of the probe. FIGS. 10A and 10B show extracellular DA levels as expressed in function of WT mice basal DA values. *p<0.05; **p<0.01, significantly different from respective basal DA values (Dunnett's multiple test). FIGS. 10C and 10D show locomotor activities before and after D-amphetamine injections. FIG. 10E shows histograms of locomotor activities for 120 minutes after the D-amphetamine injections. *p<0.05; ***p<0.001, significantly different from WT mice (Student't-test) (N=5 to 8 mice per group).

FIG. 11 is a dose response graph that demonstrates the effects of local perfusion of D-amphetamine in the nucleus accumbens on extracellular DA levels in C57BL6/J mice and Sprague-Dawley rats. Extracellular DA levels are expressed in percent of basal DA values (3.50±0.021 and 4.71±0.015 pg DA per 20 minutes for mice and rats, respectively). D-amphetamine concentrations correspond to those perfused into the probe (N=5 animals per group).

### DETAILED DESCRIPTION OF THE INVENTION

The molecular cloning of three genes encoding α1-ARs supports the existence of pharmacologically and anatomically distinct α1-AR subtypes. The α1b-receptor was originally cloned from a hamster smooth muscle cell line CDNA library, and encodes a 515 amino acid peptide that shows 42-47% homology with other ARs. The message for the α1b-receptor is abundant in rat liver, heart, cerebral cortex and kidney, and its gene was localized to human chromosome 5 (See Cotecchia et al. Proc. Natl. Acad. Sci. USA, 85, 7159-7163, 1988). A second cDNA clone from a bovine brain library was found that encodes a 466-residue polypeptide with 72% homology to the α1b-AR gene. It was further distinguished from α1b by the finding that its expression was restricted to human hippocampus, and by its localization to human chromosome 8 and it has been designated as the α1c AR (See Schwinn et al. Biol. Chem., 265, 8183-8189, 1990).

The cloning of an al α1-AR has also been reported. This gene, isolated from a rat brain CDNA library, encodes a 560-residue polypeptide that shows 73% homology with the hamster α1b-adrenergic receptor. The mRNA for this subtype is abundant in rat vas deferens, aorta, cerebral cortex and hippocampus, and its gene has been localized to human chromosome 5 (See Lomasney et al. J. Biol. Chem., 266, 6365-6369, 1991). The specific expression patterns, signaling pathways and biochemical and pharmacological properties of the α1b-AR suggests that this protein is of singular importance in the regulation of certain neurological phenomena, including addiction to drugs such as psychostimulants (e.g., D-amphetamines) and opiates.

D-amphetamine is generally assumed to exert its locomotor and rewarding effects through an increased release of dop amine (DA) in a subcortical structure, the nucleus accumbens (See Wise et al., Ann. Rev. Psychol. 19:319-40, 1996). D-amphetamine acts on both vesicular storage of DA and directly by reversing the DA transporter (DAT) located on dopaminergic terminals (See Sulzer et al., J. Neurosci. 15:4102-8, 1995). D-amphetamine acts also on noradrenergic terminals (See Nakamura et al., Neurosci. 7:2217-24, 1982) and numerous studies in mice or rats have shown that prazosin, a specific α1-adrenergic antagonist, hampers D-amphetamine-induced locomotor hyperactivity (See Darracq et al., J. Neurosci. 18:2729-39, 1998). This suggests that the stimulation of α1-adrenergic receptors is necessary to obtain D-amphetamine-induced DA release in the nucleus accumbens. However, microdialysis experiments performed in freely moving rats indicated that the inhibiting effects of prazosin on D-amphetamine-induced locomotor hyperactivity were not associated with a significant modification of the D-amphetamine-induced increase in extracellular DA levels in the nucleus accumbens (See Darracq et al., J. Neurosci. 18:2729-39, 1998). This was explained by showing that D-amphetamine-induced increase in extracellular DA levels in the nucleus accumbens could be divided into two components: a major one, due to the local effect of D-amphetamine in the nucleus accumbens and which does not cause locomotor hyperactivity (non functional DA), and a minor one, due to an effect of D-amphetamine distal from the nucleus accumbens and correlated with the development of locomotor hyperactivity (functional DA). Two sequential administrations of D-amphetamine, first a local injection into the nucleus accumbens by reverse microdialysis inducing a non functional DA release, and then a second, systemic injection, inducing locomotor hyperactivity, allowed to reach these conclusions (See Darracq et al., J. Neurosci. 18:2729-39, 1998). Pre-treatment with prazosin had no effect on non functional DA release but inhibited the functional part of the DA release, suggesting that only the minor component of the D-amphetamine-induced DA release was under the control of α1-adrenergic receptors stimulation.

Addictive drugs share the ability to stimulate dopaminergic transmission in the nucleus accumbens. However, this transmission is not the sole monoaminergic transmission involved in the behavioral effects of these compounds. Evidence based on measurement of locomotor responses to addictive drugs indicates that the noradrenergic transmission pathway is important. For example, prazosin completely inhibits locomotor hyperactivity induced by administration of D-amphetamine, cocaine, GBR12783 (a specific dopamine uptake inhibitor) and morphine. Alpha1-adrenergic transmission is also found to participate to locomotor effects of chronic D-amphetamine and cocaine use. Repeated administrations of psychostimulants potentiate the locomotor response to a subsequent drug administration, a phenomenon referred to as behavioral sensitization. When prazosin is co-administered with the repeated cocaine or D-amphetamine injections, no behavioral sensitization is observed.

Noradrenergic neurons are extremely sensitive to sensory stimulation that may exist during drug administration (handling, needle prick). It has been demonstrated that a protocol leading to a strong reaction of the animal to the injection procedure involved the noradrenergic transmission and could enhance locomotor responses to cocaine and GBR 12783. Therefore, while all drugs of abuse do not stimulate the α1b-adrenergic transmission, such a transmission is necessary for their behavioral effects, and environmental stimuli may trigger its activation.

Mice deficient in the alpha1b-adrenergic receptor have been generated and are viable, but have decreased blood pressure and vascular contractility responses. (See Cavalli et al., PNAS 94:11589-94 (1997)). The importance of the alpha1b-adrenergic receptor in certain behavioral traits has been assessed, and it has been demonstrated that the alpha1b-adrenergic receptor knock-out mouse has increased reaction to novelty, yet reduced learning capacities. (See Spreng et al., Neurobiol. Learn. Mem. 75:214-29 (2001)). Transgenic animals bearing modified alpha1b-adrenergic receptors, such as alpha1b-adrenergic receptor knock-out mice, are, therefore, useful to studying the roles of the alpha1b-adrenergic receptor in specific behaviors, including drug addiction.

To date, the mechanisms underpining the neurological basis of addiction are poorly understood. The methods and compositions according to the present invention can be used in the discovery of compounds that interact with or modulate the alpha1b-adrenergic receptor. The present invention allows the discovery of novel treatment means for addiction to psychostimulants and opiates, as well as diagnosis and treatment means for disorders associated with alpha1b-adrenergic receptor activity.

Definitions

As used herein, the term “alpha1b-adrenergic receptor”, “α-1b-adrenergic receptor” or α-1b-AR” means a molecule which is a distinct member of a class of alpha1b-adrenergic receptor molecules which under physiologic conditions, is substantially specific for the catecholamines epinephrine and norepinephrine, is saturable, and having high affinity for the catecholamines epinephrine and norepinephrine. The alpha1b-adrenergic receptor may be any mammalian alpha1b-adrenergic receptor, e.g., human (e.g., EMBL Accession Nos. NM_{—}000679 and U03865), non-human primate, or rodent (such as mouse (e.g., EMBL Accession No. Y12738), rat (e.g., EMBL Accession No. NM_{—}016991) and hamster (e.g., EMBL Accession No. J04084).

As used herein, the term “modified alpha1b-adrenergic receptor gene” means any non-wild-type alpha1b-adrenergic receptor gene, including, for example, naturally occuring or engineered allelic variants, single nucleotide polymorphisms, recombinantly generated genes, mutated genes, knock-out and knock-in genes. A modified alpha1b-adrenergic receptor gene may be a “non-functional alpha1b-adrenergic receptor gene,” such as an alpha1b-adrenergic receptor gene without detectable alpha1b-adrenergic receptor function. The term modified alpha1b-adrenergic receptor gene also includes an “alpha1b-adrenergic receptor gene having reduced function,” such as an alpha1b-adrenergic receptor gene with alpha1b-adrenergic receptor function that is detectactable but that is decreased compared to the function of a wild type alpha1b-adrenergic receptor. Mutated alpha1b-adrenergic receptors are disclosed by, e.g., Kjelsberg et al., J. Biol. Chem. 267:1430-3, 1992 and Rossier et al., Molecular Pharmacology 56:858-66, 1999).

As used herein, the term “alpha1b-adrenergic receptor activity” includes any activity now known or predicted to be associated with the alpha1b-adrenergic receptor, including, but not limited to, interactions with the catecholamines epinephrine and norepinephrine and transmission of a signal following catecholamine interaction.

As used herein, a “modulator of alpha1b-adrenergic receptor activity” is a compound that directly or indirectly changes or alters alpha1b-adrenergic receptor activity. The modulator may cause an increase in an alpha1b-adrenergic receptor activity, a decrease in an alpha1b-adrenergic receptor activity, a restoration of an alpha1b-adrenergic receptor activity such that the activity is similar to the activity of a wild-type alpha1b-adrenergic receptor, a change in locomotor response, a change in the level of extracellular dopamine in the nucleus accumbens, addiction to one or more addictive compounds, and increased or decreased conditioned place preference.

As used herein, the term “a restoration of an alpha1b-adrenergic receptor activity” refers to an increase or decrease in the level of an activity of an alpha1b-adrenergic receptor such that the increased or decreased level of this activity is similar to level of the activity of a wild-type alpha1b-adrenergic receptor.

As used herein, a “detectable phenotype” includes any chemical, biochemical, biological, physical, or behavioral event relating to a mammal or a mammalian cell, which is capable of being detected by one of ordinary skill in the art.

As used herein, a “detectable behavior” includes any behavior performed by a mammal, which can be observed and measured by one of ordinary skill in the art.

As used herein, a “candidate agent” includes any compound capable of being administered to a mammal or a mammalian cell. Suitable candidate agents include polypeptides, polypeptide fragments, nucleic acids, lipids, carbohydrates, antibodies, small molecules, peptide mimetics, hormones, small organic molecules, large organic molecules, and/or other drug candidates known to those skilled in the art, which bind or associate with an alpha1b-adrenergic receptor or a domain of the alpha1b-adrenergic receptor, particularly the α-helical bundle.

As used herein, a “peptide mimetic” is a peptide inhibitor in which one or more peptide bonds have been replaced with an alternative type of covalent bond.

As used herein, an “agonist” includes a compound that can combine with a receptor to produce a physiologic reaction. Generally, an agonist increases the activity of a receptor.

As used herein, an “inverse agonist” includes a compound that acts at the same receptor as that of an agonist, yet produces an opposite effect, and includes a negative antagonist. Inverse agonists of the alpha1b-adrenergic receptor are also known as alpha-blockers or α-blockers.

As used herein, an “antagonist” includes a compound that interferes with the physiological action of another molecule or other compound e.g., a receptor, such as by binding to the receptor and preventing or interfering with the binding of a ligand or an agonist to the receptor.

As used herein, an “alpha1b-adrenergic receptor-associated disorder” includes any disorder, disease, pathology, or abnormality that is initiated by or progresses due to one or more activities of an alpha1b-adrenergic receptor.

As used herein, an “inducer of an alpha1b-adrenergic receptor-associated disorder” includes any compound or event that increases the likelihood that a disease or disorder associated with the activity of the alpha1b-adrenergic receptor will occur, and/or any compound or event that increases the progression of a disease or disorder associated with the activity of the alpha1b-adrenergic receptor.

As used herein, a “repressor of an alpha1b-adrenergic receptor-associated disorder” includes any compound or event that decreases the likelihood that a disease or disorder associated with the activity of the alpha1b-adrenergic receptor will occur, and/or any compound or event that decreases or eliminates the progression of a disease or disorder associated with the activity of the alpha1b-adrenergic receptor.

As used herein, the term “reference response” includes a response derived from a previously measured response from a mammal or a mammalian cell. In some embodiments, the reference response is derived from the detected responses of two or more mammalian cells.

As used herein, the term “drug addiction” includes any biochemical, physical, or mental reliance or dependence of a mammal on a compound.

As used herein, the term “a compound that promotes addiction” includes any compound that initiates or continues a biochemical, physical or mental reliance or dependence of a mammal.

The invention provides methods of identifying a modulator of alpha1b-adrenergic receptor activity by administering a candidate agent to a first mammal containing a modified alpha1b-adrenergic receptor gene, measuring a detectable phenotype of the first mammal, and comparing the detectable phenotype of the first mammal with a detectable phenotype of a second mammal. An alteration in the detectable phenotype of the first mammal compared to the second mammal indicates that the candidate agent is a modulator of alpha1b-adrenergic receptor activity. The first mammal can be one of any mammalian species capable of being recombinantly altered to contain a modified alpha1b-adrenergic receptor gene. Rats, mice and other rodents are preferred mammals.

The invention further provides methods of identifying a modulator of alpha1b-adrenergic receptor activity by administering a candidate agent to a test mammalian cell containing a modified alpha1b-adrenergic receptor gene, measuring a detectable response by the test mammalian cell, and comparing the detectable response of the test mammalian cell with a reference response. A change in the detectable response of the test mammalian cell relative to the reference response indicates that the candidate agent is a modulator of alpha1b-adrenergic receptor activity. The detectable response may be a change in the level of dopamine secreted by a mammalian cell. The reference response may be a response derived from a mammalian cell containing a wild-type alpha1b-adrenergic receptor.

The invention also provides methods of identifying a compound that inhibits or reduces drug addiction by providing a first mammal containing a modified alpha1b-adrenergic receptor gene, administering a candidate agent to first mammal, measuring a detectable behavior correlated with an addiction of the first mammal, and comparing the detectable behavior of the first mammal with a detectable behavior of a second mammal, which does not have a modified alpha1b-adrenergic receptor gene. A decrease in the detectable behavior of the second mammal relative to the first mammal indicates that the candidate agent is a compound that inhibits or reduces addiction. The modified alpha1b-adrenergic receptor gene may be a non-functional alpha1b-adrenergic receptor gene or an alpha1b-adrenergic receptor gene with reduced function as compared to a wild-type alpha1b-adrenergic receptor gene. The candidate agent may be an antagonist or inverse agonist selective for the alpha1b-adrenergic receptor.

The invention further provides methods for identifying a compound that promotes addiction by providing a first mammal, which contains a modified alpha1b-adrenergic receptor gene that is either a non-functional alpha1b-adrenergic receptor gene or an alpha1b-adrenergic receptor gene with reduced function as compared to a wild-type alpha1b-adrenergic receptor gene, administering a candidate agent to the first mammal, measuring a detectable behavior correlated with an addiction of the first mammal, and comparing the detectable behavior of the first mammal with a detectable behavior of a second mammal, which does not have a modified alpha1b-adrenergic receptor gene. An increase in the detectable behavior of the second mammal relative to the first mammal indicates that the candidate agent is a compound that promotes addiction. This compound can be any drug or other entity that promotes addiction. For example, psychostimulants and opiates are preferred compounds.

The invention also provides methods of identifying an inducer of an alpha1b-adrenergic receptor-associated disorder by providing a first mammal, which contains a modified alpha1b-adrenergic receptor gene, administering a candidate agent to said first mammal, measuring a detectable phenotype of the first mammal, and comparing the detectable behavior of the first mammal with a detectable behavior of a second mammal, which does not have a modified alpha1b-adrenergic receptor gene. An increase in the detectable behavior of the second mammal relative to the first mammal indicates that the candidate agent is an inducer of an alpha1b-adrenergic receptor-associated disorder.

The invention provides methods of identifying a repressor of an alpha1b-adrenergic receptor-associated disorder, by providing a first mammal containing a modified alpha1b-adrenergic receptor gene, administering a candidate agent to the first mammal, measuring a detectable phenotype of the first mammal, and comparing the detectable behavior of the first mammal with a detectable behavior of a second mammal that does not have a modified alpha1b-adrenergic receptor gene. A decrease in the detectable behavior of the second mammal relative to the first mammal indicates that the candidate agent is a repressor of an alpha1b-adrenergic receptor-associated disorder.

Transgenic mammals with modified alpha1b-adrenergic receptors.

The present invention relates, in part, to methods using transgenic animal systems having a modified alpha1b-adrenergic receptor, such as a transgenic mouse lacking a functional alpha1b-adrenergic receptor. One non-limiting example describing the generation of a mouse deficient for the alpha1b-adrenergic receptor is described by Cavalli et al. (See Cavalli et al., PNAS 94:11589-94 (1997)).

Generally, the preparation of a transgenic mammal requires introducing a nucleic acid construct that will be used to express a nucleic acid encoding a modified alpha1b-adrenergic receptor into an undifferentiated cell type, e.g., an embryonic stem (ES) cell. The ES cell is then injected into a mammalian embryo, where it will integrate into the developing embryo. The embryo is then implanted into a foster mother for the duration of gestation.

Embryonic stem cells are typically selected for their ability to integrate into and become part of the germ line of a developing embryo so as to create germ line transmission of the heterologous gene construct. Thus, any ES cell line that has this capability is suitable for use herein. One mouse strain that is typically used for production of ES cells is the 129/Sv strain. A preferred ES cell line is murine cell line HM-1. The cells are cultured and prepared for DNA insertion using methods well known in the art, such as those set forth by Robertson (Robertson, In: Teratocarcinomas and Embryonic Stem Cells: A Practical Approach, E. J. Robertson, ed., IRL Press, Washington, D.C., 1987.). Insertion of the nucleic acid construct into the ES cells can be accomplished using a variety of methods well known in the art including for example, electroporation, microinjection, and calcium phosphate treatment.

The term “transgene” is used herein to describe genetic material that has been or is about to be artificially inserted into the genome of a mammalian cell, particularly a mammalian cell of a living animal. The transgene is used to transform a cell, meaning that a permanent or transient genetic change, preferably a permanent genetic change, is induced in a cell following incorporation of an heterologous nucleic acid, such as DNA. A permanent genetic change is generally achieved by introduction of the DNA into the genome of the cell. Vectors for stable integration include plasmids, retroviruses and other animal viruses, yeast artificial chromosomes (YAC)s, and the like. Transgenic mammals, may include, e.g. cows, pigs, goats, horses, and particularly rodents, e.g., rats and mice. Preferably, the transgenic animals are mice.

Transgenic animals have a heterologous nucleic acid sequence present as an extrachromosomal element or stably integrated in all or a portion of its cells, especially in germ cells. Unless otherwise indicated, it is assumed that a transgenic animal comprises stable changes to the germline sequence. During the initial construction of the animal, “chimeras” or “chimeric animals” are generated, in which only a subset of cells have the altered genome. Chimeras are primarily used for breeding purposes in order to generate the desired transgenic animal. Animals having a heterozygous alteration are generated by breeding of chimeras. Male and female heterozygotes are typically bred to generate homozygous animals.

The heterologous gene is usually either from a different species than the animal host, or is otherwise altered in its coding or non-coding sequence. The introduced gene may be a wild-type gene; naturally occurring polymorphism; or a genetically manipulated sequence, for example a sequence having deletions, substitutions or insertions in the coding or non-coding regions. Where the introduced gene is a coding sequence, it is usually operably linked to a promoter, which may be eitherconstitutive or inducible, and other regulatory sequences required for expression in the host animal. By “operably linked” is meant that a DNA sequence and a regulatory sequence(s) are connected in such a way as to permit gene expression when the appropriate molecules, e.g., transcriptional activator proteins, are bound to the regulatory sequence(s).

The transgenic animals of the invention may contain other genetic alterations in addition to the presence of the heterologous gene. For example, the host's genome may be altered in order to affect the function of endogenous genes (e.g., functionally inactive alpha1b-adrenergic receptors or alpha1b-adrenergic receptors with reduced function), may contain marker genes, or may have other genetic alterations.

The transgenic animals described herein may contain alterations to endogenous genes. For example, the host animals may be either “knockouts” and/or “knockins” for a target gene(s). Specifically, the host animal's endogenous alpha1b-adrenergic receptor may be “knocked out” and/or the a modified alpha1b-adrenergic receptor “knocked in”. Knockouts have a partial or complete loss of function in one or both alleles of an endogenous gene of interest (e.g., alpha1b-adrenergic receptor). Knockins have an introduced transgene with altered genetic sequence and/or function from the endogenous gene. Knockouts and knockins may be combined, for example, such that the naturally occurring gene is disabled, and an altered form introduced. For example, it may be desirable to knockout the host animal's endogenous alpha1b-adrenergic receptor gene, while also introducing a modified alpha1b-adrenergic receptor gene.

Preferably, the target gene expression in a knockout is undetectable or insignificant. For example, a knock-out of an alpha1b-adrenergic receptor gene means that function of the alpha1b-adrenergic receptor has been substantially decreased so that expression is not detectable or only present at insignificant levels. This may be achieved by any means known to those skilled in the art, including introduction of a disruption of the coding sequence, e.g., insertion of one or more stop codons, insertion of a DNA fragment, etc.; deletion of coding sequence; substitution of stop codons for coding sequence, etc. In some cases, the exogenous transgene sequences are ultimately deleted from the genome, leaving a net change to the native sequence. Different approaches may be used to achieve the “knockout”. For example, a chromosomal deletion of all or part of the native gene may be induced, including deletions of the non-coding regions, particularly the promoter region, 3′ regulatory sequences, enhancers, or deletions of gene that activate expression of alpha1b-adrenergic receptor genes. A functional knockout may also be achieved by the introduction of an anti-sense construct that blocks expression of the native genes (See, e.g., Li and Cohen (1996) Cell 85:319-329). “Knockouts” also include conditional knockouts, for example, knockouts where alteration of the target gene occurs upon exposure of the animal to a substance that promotes target gene alteration, introduction of an enzyme that promotes recombination at the target gene site (e.g. Cre in the Cre-lox system), or other method for directing the target gene alteration postnatally.

A “knockin” of a target gene refers to an alteration in a host cell genome that results in altered expression or function of a native target gene. Increased (including ectopic) or decreased expression may be achieved by introduction of an additional copy of the target gene, or by operatively inserting a regulatory sequence that provides for enhanced expression of an endogenous copy of the target gene. These changes may be either constitutive or conditional, i.e. dependent on the presence of an activator or represser. The use of knockin technology may be combined with production of exogenous sequences to produce the transgenic animals of the invention.

Heterologous gene constructs may include a nucleic acid encoding a alpha1b-adrenergic receptor protein. The heterologous gene construct can also encode for various accessory proteins required for the functional expression of the alpha1b-adrenergic receptor protein, as well as for selection markers and enhancer elements.

A selection marker can be any nucleic acid sequence that is detectable and/or assayable. Examples of selection markers include positive selection markers and negative selection markers. Positive selection markers include drug resistance genes; (e.g., neomycin resistance genes or hygromycin resistance genes), or beta-galactosidase genes. Negative selection markers, e.g., thymidine kinase gene, diphtheria toxin gene and ganciclovir, are useful in the heterologous gene construct in order to eliminate embryonic stem (ES) cells that do not undergo homologous recombination. The selection marker gene is usually operably linked to its own promoter or to another strong promoter from any source that will be active or can easily be activated in the cell into which it is inserted. However, the marker gene need not have its own promoter attached, as it may be transcribed using the promoter of the alpha1b-adrenergic receptor gene to be suppressed. In addition, the marker gene will normally have a polyA sequence attached to the 3′ end of the gene, which serves to terminate transcription of the gene.

“Enhancer elements” include, for example, nucleic acid sequences that are bound by polypeptides associated with transcription, and are usually in cis with the nucleic acid encoding an alpha1b-adrenergic receptor protein. Examples of enhancer elements include cyclic AMP response elements (CRE), serum response elements (SRE), nuclear factor B (NF-KB), activator protein 1 (AP-1), serum response factor (SRF), and p53 binding sites. These enhancer elements may further include a TATA box.

The heterologous gene construct may be constituitively expressed in the transgenic mammal. The gene construct may expressed in specific tissues, e.g., the construct is under the control of a tissue-specific promoter.

The gene construct can be under the control of an inducible (activatible) promoter. Activation of the promoter results in increased expression of the gene construct encoding the alpha1b-adrenergic receptor proteins and the accessory proteins, if present. Similarly, repression of an inducible promoter results in decreased expression of the gene construct encoding the alpha1b-adrenergic receptor proteins and accessory proteins. Activation of the promoter is achieved by the interaction of a selected biocompatible entity, or parts of the entity, with the promoter elements. Promoter activation also includes the administration of a substance that stimulates production of an endogenous promotor activator, and the imposition of conditions resulting in the production of an endogenous promotor activator (e.g., heat shock, stress). If the activation occurs only in a part of the animal, only cells in that part will express the alpha1b-adrenergic receptor protein.

Administration of Candidate Agents

The candidate agent of the invention can be administered to a mammal by methods generally known to those skilled in the art of drug delivery. The administration of the candidate agent is usually by oral administration or parenteral administration. Parenteral administration includes, for example, subcutaneous administration, subdermal administration, intraarterial administration, intravenous administration, intraperitoneal administration, topical administration, ophthalmic administration, nasal administration, and intramuscular administration.

The candidate agents of the invention (also referred to herein as “active compounds”) of the invention, and derivatives, fragments, analogs and homologs thereof, can be incorporated into pharmaceutical compositions suitable for administration. Such compositions typically comprise the compound, nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include; but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The candidate agent of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. “Dosage unit form,” as used herein, refers to physically discrete units suited as unitary dosages for the subject to be treated. Each unit may contain a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Administration of a candidate agent or other compound to a mammalian cell can be performed by any method known to one of ordinary skill in the art of mammalian cell culture. Administration of a candidate agent or other compound to a mammalian cell includes, for example, direct injection of the agent or compound into the mammalian cell (e.g., by microinjection), by contacting the agent or compound with the cell culture media contacting the mammalian cell, or treating a surface or other material with an agent (such as by adsorption) and contacting the agent-treated surface with the mammalian cell.

Candidate Agents

The invention provides candidate agents that can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. Typically, the biological library approach is limited to peptide libraries, while the other approaches are applicable to peptide, non-peptide oligomer or small molecule libraries of compounds. See, e.g., Lam, 1997. Anticancer Drug Design 12: 145.

Suitable candidate agents include polypeptides, polypeptide fragments, nucleic acids, lipids, carbohydrates, antibodies, small molecules, peptide mimetics, hormones, small organic molecules, large organic molecules, and/or other drug candidates known to those skilled in the art, which bind or associate with an alpha1b-adrenergic receptor or a domain of the alpha1b-adrenergic receptor, particularly the α-helical bundle. Preferred candidate agents are agonists, inverse agonists, and antagonists of the alpha1b-adrenergic receptor. Inverse agonists include quinazolines (e.g., prazosin, terazosin, alfuzosin, and molecules of similar structure), and N-arylpiperazines (e.g., REC 15/3039, REC 15/2739, and REC 15/3011, which are specific inverse agonists for the alpha1b-adrenergic receptor). (See, e.g., Rossier et al., Molecular Pharmacology 56:858-66, 1999). Candidate agents can be generated by molecular modeling of ligands, such as by using MOPAC 6.0 (QCPE 445) program and the QUANTA molecular modeling package (Molecular Simulation, Inc., Waltham, Mass.).

Libraries of chemical and/or biological mixtures, such as fungal, bacterial, or algal extracts, are known in the art and can be screened with any of the assays of the invention. Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409).

Detectable Phenotypes

Any phenotype exhibited by the mammal to which a candiate agent has been administered that is capable of detection may be used in the present invention. Such phenotypes include an increase in an alpha1b-adrenergic receptor activity, a decrease in an alpha1b-adrenergic receptor activity, a restoration of an alpha1b-adrenergic receptor activity such that the activity is similar to the activity of a wild-type alpha1b-adrenergic receptor, a locomotor response, the level of extracellular dopamine in the nucleus accumbens, fluid consumption, addiction to one or more addictive compounds, and conditioned place preference. These phenotypes are described in detail in the Examples section and in, e.g., Spreng et al., Neurobiol. Learn. Mem. 75:214-29 (2001); and Auclair et al., J. Neurosci. 22:9150-4 (2002).

Detectable Responses

A detectable response includes any internal or external biochemical, biological or chemical change, exhibited by a mammalian cell in response to administration of a candidate agent, that is capable of being detected. For example, a detectable response may be change in the level of dopamine secreted by the test mammalian cell. Other detectable responses include changes in pH, electrical charge of the cell, cell polarity, exocytosis or endocytosis, proliferation, cell motility, metabolism and/or catabolism, DNA and/or amino acid synthesis; and apoptosis or necrosis.

A reference response includes a response derived from a previously measured response from a mammal or a mammalian cell. The reference response may be derived from the detected responses of two or more mammalian cells. The reference response may be derived from the test mammalian cell containing a modified alpha1b-adrenergic receptor, or, alternatively, from a mammalian cell containing a wild-type alpha1b-adrenergic receptor.

Detectable Behaviors

Any behavior exhibited by the mammal to which a candiate agent has been administered that is capable of detection may be used in the present invention. Such behaviors include, e.g., a locomotor response, fluid consumption, addiction to one or more addictive compounds, conditioned place preference, spatial learning ability, memory, and stress. These behaviors are described in detail in the Examples section and in, e.g., Spreng et al., Neurobiol. Learn. Mem. 75:214-29 (2001); and Auclair et al., J. Neurosci. 22:9150-4 (2002).

Use of Identified Compounds in Pharmaceutical Compositions

The invention further pertains to agents identified using the methods of the invention as well as uses thereof in pharmaceutical compositions for treatments as described herein. The pharmaceutical compositions of the invention include the novel agents identified using the methods of the invention combined with a pharmaceutically acceptable carrier. The term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

### EXAMPLES

The invention will be further described in the following examples, which do not limit the scope of the invention described in the claims.

### Example 1

### General Materials and Methods

Animals.

Mice.

Animals were adult male mice bred at the “Institut de Pharmacologic et Toxicologie” (Lausanne, Switzerland), weighting 35-45 g, when experiments took place. The genetic background of the mice was a 129/SvXC57BL/6J mixture for both the WT and α1b-AR KO mice. (See Cavalli et al., PNAS 94:11589-94 (1997)). Two out of seven chimerical mice, which were mated, gave rise to germ line transmission of the disrupted allele generating heterozygous mice. Heterozygous mice were mated to obtain the homozygous α1b-AR+/+ (WT) and −/− (KO) progeny. For each genotype, mice from different litters were randomly inter-crossed to obtain the WT and KO progeny used in this study. Since 1997, at least forty inter-crosses have occurred. The mice were never inter-crossed with other strains or mated with those from the same litters. Animal experimentation was conducted in accordance with the guidelines for care and use of experimental animals of the European Economic Community (86/809; DL27.01.92, Number 116).

Rats.

Male Sprague Dawley rats (Iffa-Credo, Lyon, France) were used as subjects in reverse dialysis experiments. They weighted 280-300 g at the time of surgery. All animals were housed in plastic cages with food and water provided ad libitum. Colony rooms were maintained under constant temperature and humidity on a 12-h light/dark cycle (07:00 to 19:00). Experimentations were conducted in accordance with the guidelines for care and use of experimental animals of the European Economic Community (86/809; DL27.01.92, Number 16). All efforts were made to minimize the number of animals used and their suffering.

Surgery.

Mice were anaesthetised with sodium pentobarbital (60 mg/kg; Sanofi Santé Animale, France) and placed in a stereotaxic frame (Kopf instruments). The head was positioned by means of a mouse nose clamp adaptator (Kopf model 922) supplemented by rat ear bars placed lightly in the external auditory meatus. A unilateral permanent cannula (CMA/7 guide cannula, Microdialysis AB, Sweden) was implanted into the nucleus accumbens and was secured on the skull with screw and dental cement. The coordinates for the guide cannula tip were antero-posterior (AP): +1.3 relative to bregma, medio-lateral (ML): +0.8, and dorso-ventral (DV): −2,4 mm from dura (Paxinos and Franklin, The mouse brain in stereotaxic coordinates, Academic Press 2001).

Rats were anesthetised with sodium pentobarbital (60 mg/kg; Sanofi Santé Animale, France). A unilateral permanent cannula (CMA/11 guide cannula, Microdialysis AB, Sweden) was implanted into the nucleus accumbens. The coordinates for the guide cannula tip were antero-posterior (AP): +1.7 relative to bregma, medio-lateral (ML): +1.1, and dorso-ventral (DV): −5.7 mm from dura (Paxinos and Watson, Ann. Rev. Pharmacol. Toxicol. 32:63(−77, 1986). After surgery, animals were placed in individual plastic cages and allowed to recover for at least 4 days.

Drugs.

D-amphetamine sulfate, cocaine hydrochloride, scopolamine hydrobromide and chloro-APB hydrobromide were purchased from Sigma (L'isle d'Abeau, France) and morphine chiorhydrate from Francopia (Paris, France). Doses are expressed as salts.

Locomotor Activity.

Mice were introduced in a circular corridor (4.5 cm width, 17 cm external diameter) crossed by four infrared beams (1.5 cm above the base) placed at every 90° (Imetronic, Pessac, France). The locomotor activity was counted when animals interrupted two successive beams and, thus, had traveled ¼ of the circular corridor. In each session, the spontaneous activity was recorded for 90 minutes, before mice received either saline or drugs. Their activity was recorded for an additional 60 minutes or 120 minutes period. The first session was a session of habituation to the experimental procedure during which animals received saline (˜3 ml/kg, i.p.). The locomotor response to a drug administration was measured on the following day. Tests were performed between 12:00 and 6:00 p.m. in stable conditions of temperature and humidity.

Autoradiography.

Brains were rapidly removed after animal death and frozen in isopentane (−30° C.). Sections (20 μM) were cut with a cryostat, mounted onto gelatin-coated glass slides and stored at −20° C. until incubation. For Dl binding sites, sections were incubated with H-SCH23390 as previously described (Trovero et al., J. Neurochem. 59:331-7, 1992). For D2 binding sites, sections were incubated for 60 minutes at 20° C. in Tris-HCl buffer (50 mM, pH 7.4) containing 0.2 nM ¹²⁵-iodosulpride (NEN Dupont, France), washed 5 times in ice-cold Tris-HCl buffer (50 mM, pH 7.4), dried and exposed to ³H-hyperfilm for 10 days. For DAT binding sites, sections were incubated for 2 hours at 20° C. in NaH₂PO₄ buffer (50 mM, pH 7.4) containing 7.5 mM 3H-W1N35,428 (NEN Dupont, France), washed 2 times in ice-cold NaH₂PO₄ buffer (50 mM, pH 7.4), dried and exposed to ³H-hyperfihn for 15 days. For VMAT binding sites, sections were incubated for one hour at 20° C. in HEPES buffer (20 mM, pH 8.0) containing 0.3 M sucrose and 2 nM ³H-dihydrotetrabenazine (Amersham, France), washed 2 times in ice-cold Tris-HCl buffer (50 mM, pH 7.4), rinsed in distilled water, dried and exposed to ³H-hyperfilm for 2 months. Autoradiograms were digitised and quantified with a video-imager (ImageQuest video software)

Histology.

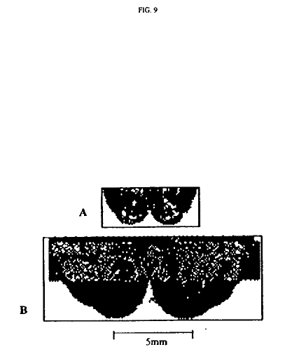

At the end of the experiment, brains of mice or rats were conserved into formaldehyde solution and cut on a microtome in serial coronal slices according to the atlas of Franklin and Paxinos (2001) (mice) or Paxinos and Watson (1986) (rats). Histological examination of cannula tip placement was subsequently made on 100-μm safranine stained coronal sections (FIG. 9).

Monoamine Tissue Contents.

Brains were rapidly extracted after animal death, split into two parts in a frontal plane. Anterior parts were frozen in dry ice. Tissue samples were punched out from frontal slices (300 μm obtained with a microtome refrigerated at −12° C.) with cooled stainless tubes (an equilateral triangular shape of 3.7 mm side for both sides of prefrontal cortex, circular shape of 0.9 mm diameter for nucleus accumbens and of 1.4 mm diameter for striatum). Samples were dissolved and sonicated in 150 μL of perchloric acid (0.1 N), sodium metabisulfite (0.05%). After centrifugation, supernatants were used to simultaneously estimate DOPAC, DA and NE via a column of HPLC coupled to electrochemical detection previously described (Vezina et al, J. Pharmacol. Exp. Ther. 261:484-90, 1992). Protein quantities were determined from the pellets with the bicinchoninic acid—based method (Smith et al., Anal. Biochem. 150:76-85, 1985).

Adenylyl Cyclase Assay in Vitro.

Four microdisks (diameter, 0.9 mm) were punched bilaterally from the central striatum, blown into 200 μl of 1 mM Tris-maleate (pH 7.2), containing 2 mM EGTA (pH 7.2) and 300 mM sucrose, and gently homogenized in a Potter Elvebjem apparatus (10 strokes). Adenylyl cyclase activity was assayed by measuring conversion of [α-³²P}ATP into [α-³²P]cyclic AMP in the presence or absence of 10⁻⁴ M DA. [α-³²P}cyclic AMP was purified according to Salomon et al (1974). Adenylyl cyclase activity was expressed as pmole of cyclic AMP produced per min and mg protein.

Oral Consumption.

Fluid intake was measured daily by weighting bottles, mice being housed individually. Tested solutions were replaced twice weekly.

Cocaine and morphine: For two weeks, bottles were filled with cocaine or morphine solution of decreasing concentration (one dose per week; 0.3 mg/ml then 0.2 mg/ml for cocaine and 0.2 mg/ml and 0.15 mg/ml for morphine) instead of water, so that mice accustom to the bitter taste. Replacing water by cocaine produced no significant alteration of mice fluid intake. Replacing water by morphine significantly increased WT fluid intake (97.5±5.0 ml/kg/day for water vs. 117.2±3.9 for morphine (0.15 mg/ml), p<0.001; paired Student's t-test), but not α1b-AR KO fluid intake. Then, to determine mean water consumption and eliminate basal side preference, two bottles of water were given for three days to each animal and basal consumption was calculated as the mean consumption from one bottle on day 2 and the other on day 3. Preference for cocaine (0.2 mg/ml) or morphine (0.15 mg/ml) was then measured over two 12-day sessions, drug and water sides being exchanged between the 2 sessions. Means of drug and water consumptions were estimated for the last 5 days of both 12-day periods.

Sucrose and quinine: In this case, mice were first exposed for three days to two bottles of water to measure basal preference as described above. Then on one side, the bottle was filled with either quinine or sucrose solution and bottles were exchanged on the following day, the mean consumptions of water and either sucrose or quinine being estimated on a two-day period. Several concentrations of either quinine or sucrose were tested with the same mice in random order.

Morphine-Induced Conditioned Place Preference.

Conditioned place preference was measured in a Y maze. Mice were habituated to the experimental apparatus (Imetronic, Pessac, France) for 3 days (1 hour of exploration of the maze with neutral cues, i.e. smooth gray walls and floor). On the following day, animals were allowed to freely explore the two compartments (with different visual and tactile cues) of the maze for 20 minutes corresponding to a pre-conditioning test. The total amount of time spent in each compartment was recorded and analyzed as previously described (Valverde et al., Psychopharmacology (Berlin) 123:119-26, 1996). For the next 8 days corresponding to the conditioning session, mice alternatively received morphine (5 mg/kg; s.c.) in one compartment and saline in the other compartment the following day. According to the unbiased method, morphine was equally associated to both compartments and was given either first or second, mice being confined in one compartment for the 30 minutes following each injection. One day after the end of the conditioning session, mice were submitted to a post-conditioning test identical to the test performed before conditioning, i.e. each mice being allowed to explore both compartments for 20 minutes. A four day conditioning session was added and a second post-conditioning test was performed similar to the first one. Locomotor activities were recorded with electronic cells in each conditioning session.

Microdialysis Experiments.

The day of the experiment, the microdialysis probe was inserted (CMA/7, Membrane length 2 mm and diameter 0.24 mm, cut-off: 6000 Da, Microdialysis AB, Sweden, for mice or CMA/11 with identical probe characteristics for rats). Artificial CSF (in mM: NaCl: 147; KCl: 3.5; CaCl₂: 1; MgCl₂: 1.2; NaH₂PO₄: 1; NaHCO₃: 25, pH=7.6) was perfused with a CMA100 microinjection pump through the probe at a rate of 1 μl/min (2 μl/min for rats) via FEP catheter (internal diameter 0.12 mm for mice) or polyethylene catheter (internal diameter 0.3 mm for rats) connected to a fluid swivel. Adequate steady state of DA levels in perfusate samples was reached 2 hours and 20 minutes after probe insertion for mice and rats, and samples were collected in 300 μl vials placed into a refrigerated computer-controlled fraction collector (CMA/170). Samples (20 μl every 20-minutes for mice and 10 μl every 5 minutes for rats) were collected during 100 minutes and 30 minutes for mice and rats, respectively, to determine basal extracellular DA values. Following D-amphetamine injection, samples were collected for 2 hours and 40 minutes. For reverse dialysis experiments, D-amphetamine (3, 5, 10 and 100 μM) was infused during one hour after determination of the extracellular basal DA level.

Biochemistry.

Dialysate samples were completed to 30 μl with the mobile phase and placed into a refrigerated automatic injector (Triathlon, Spark Holland, Emmen, The Netherlands). Twenty five μl of the sample were injected every 15 minutes through a rheodyne valve in the mobile phase circuit. High-performance liquid chromatography was performed with a reverse-phase column (80×4.6 mm, 3 μM particle size, HR-80, ESA INC., Chelmsford, Mass.). Mobile phase (NaH₂PO₄ 75 mM, EDTA 20 μM, octane sulfonic acid 2.75 mM, triethylamine 0.7 mM, acetonitrile 6%, methanol 6%, and pH 5.2) was delivered at 0.7 ml/min by an ESA-580 pump. Electrochemical detection was performed with an ESA coulometric detector (Coulochem II 5100A, with a 5014B analytical cell; Eurosep, Cergy, France). The conditioning electrode was set at −0.175 mV, and the detecting electrode was set at +0.175 mV, allowing a good signal-to-noise ratio of the DA oxidation current. External standards were regularly injected to determine the stability of the sensitivity (0.3-0.4 pg of DA).

Data Analysis.

Data were analyzed with Student's t-test or analysis of variance. For behavioral sensitization experiments, correlation between the number of drug injections and the amplitude of the locomotor responses was analyzed with linear regression and the influence of genotype on this correlation with an analysis of covariance. Genotype and prazosin treatments were between-subjects factors. Time, sucrose and quinine concentration, chloro-APB doses, number of injections (for behavioral sensitization and morphine-induced CPP) were within subjects factors. Differences were considered significant when p<0.05.

Statistics.

Results presented are means±S.E.M of data obtained with 5 to 9 animals. Statistical analysis was performed using Graph Pad Prism 3.0 software, (San Diego, Calif.). Data from microdialysis experiments were expressed as a percentage of the respective mean basal value to compensate for inter-subject differences. The extracellular DA levels obtained before and after the D-amphetamine intraperitoneal injection (3 and 6 mg/kg,) were compared and analysed with repeated measures ANOVA (two-way and one-way ANOVA followed by a Dunnett's multiple comparison test). Locomotor activities following D-amphetamine were compared to the locomotor basal activity with a two-way ANOVA and between doses with a Student's t-test. The effects of the concentration of local D-amphetamine and of rodent species on the increase in extracellular DA levels were tested with a two way ANOVA. LogEC50's were compared after fitting curves with a Student's t-test. Pharmacological treatments correspond to independent groups of animals. Significant differences were set at p<0.05.

### EXAMPLE 2

### Control of Locomotor and Rewarding Effects of Psychostimulants and Opiates by alpha1b-Adrenergic Receptors

Prazosin Binding Sites in the Brain of WT and α 1b-AR KO Mice

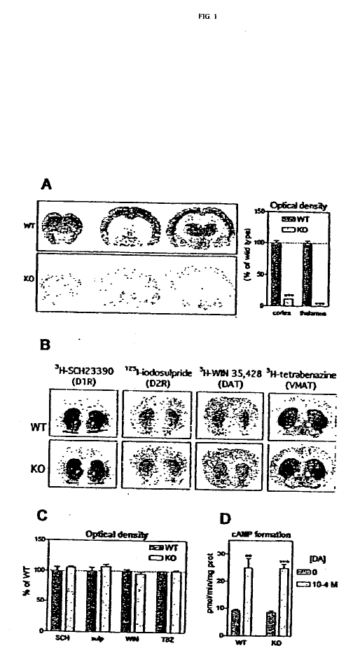

Distributions of ³H-prazosin binding sites on coronal brain sections were compared between WT and α1b-AR KO mice (FIG. 1A). ³H-prazosin binding pattern in WT brains was similar to those previously described (Trovero et al., Neurosci. 47:69-76, 1992), with particularly high densities in the layer III of the cerebral cortex and in the thalamus. For α1b-AR KO mice, binding densities were dramatically decreased in these regions (−88% in cortical layer III and −97% in thalamus, compared to WT, p<0.001, Student's t-test) and the typical pattern of prazosin binding was lost.

Equivalent Basal Dopaminergic Transmission in the Brains of WT and α 1b AR Mice

Striatal distributions (FIG. 1B) and densities (FIG. 1C) of D1 and D2 DA receptors, as well as DA and vesicular monoamine transporters measured by autoradiography with specific radioactive ligands revealed no significant difference between WT and α1b-AR KO animals. Furthermore, the sensitivity of striatal D1 DA receptor to DA measured by in vitro adenylyl cyclase assay was unaltered in a1b-AR KO mice (FIG. 1D). Finally, tissue contents of NE, DA and DOPAC in the prefrontal cortex, nucleus accumbens and striatum of WT and α1-AR KO mice were equivalent (Table 1). DOPAC/DA ratios were unmodified, suggesting that basal DA utilization was the same in WT and α1b-AR KO brains.

Equivalent Locomotor Responses to Novelty, Saline Injection, Scopolamine and Chloro-AFB in WT and α1b-AR KO Mice

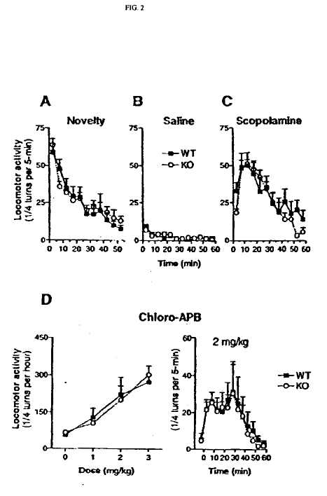

No significant difference was observed between WT and α1b-AR KO mice when their locomotor activity was recorded immediately after their first introduction in the experimental apparatus (time×genotype: F_{10,220}=0.92, p=0.52; genotype: F_{1,220}=0.03, p=0.86, two-way RM Anova) (FIG. 2A).

Furthermore, basal locomotor responses of WT and α1b-AR KO mice to an intraperitoneal saline injection were similar (FIG. 2B) (time×genotype: F_{1,242}=1.21, p=0.283 and genotype: F_{1,242}=0.01, p=0.92, two-way RM Anova).

The stimulatory effect of scopolamine (1 mg/kg, i.p.), a centrally acting muscarinic antagonist known to act independently from catecholaminergic transmission (Blanc et al., Eur. J. Neurosci. 6:293-8, 1994) (FIG. 2C) were equivalent in WT and α1b-AR KO mice (time×genotype: F_{1,209}=1.63, p=0.092 and genotype: F_{1,209}=0.32, p=0.58, two-way RM Anova).

Finally, chloro-APB, a D1 receptor agonist, dose-dependently increase locomotor activity of WT mice (F_{3,21}=9.08, p<0.001, one-way RM Anova). Similar effects are observed in α1bAR KO mice (F_{3,21}=18.7, p<0.0001, one-way RM Anova). No significant differences are observed in the amplitudes of locomotor responses to chloro-APB between WT and α1b-AR KO mice (dose×genotype: F_{3,28}=0.66 p=0.58, genotype: F_{1,28}=0.0 p=0.95, two-way RM Anova; FIG. 2D).

Altogether, these data suggested that α1b-AR KO mice are devoid of gross neurological deficits and are therefore suitable to analyze the role of α1b-ARs in the responses to psychostimulants and opiates.

Reduced Locomotor Response of α 1b-AR 10 Mice to D-Amphetamine, Cocaine and Morphine

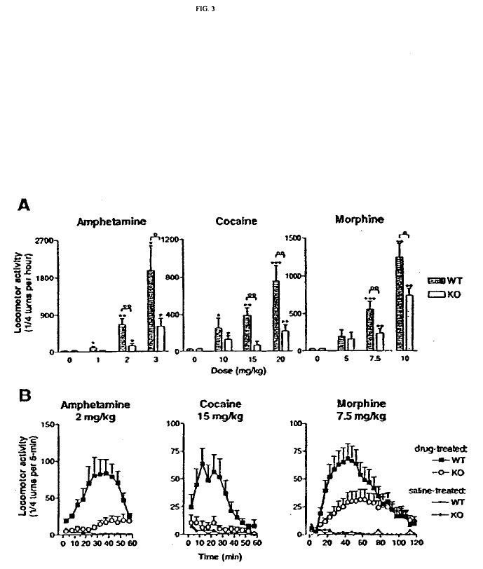

D-amphetamine, cocaine and morphine induce a dose-dependent stimulation of locomotor activity in WT mice (D-amphetamine: F_{3,30}=14.9 p<0.0001; cocaine: F_{3,33}=9.3 p=0.0001; morphine F_{3,30}=19.0 p<0.0001; one-way Anova) (FIG. 3A). In α1b-AR KO mice, these drugs also increased locomotor activity (D-amphetamine: F_{3,32}=9.19 p<0.001; cocaine: F_{3,35}=3.65 p<0.05; morphine F_{3,32}=14.0, p<0.0001; one-way Anova). However, amplitudes of locomotor responses were significantly lower in α1b-AR KO mice compared to WT animals.

For D-amphetamine, amplitude of locomotor response was significantly altered by genotype, depending on the dose of D-amphetamine (genotype×dose: F_{2,47}=5.63 p<0.05, genotype: F_{1,47}=17.26, p<0.0001; two-way Anova) and locomotor activity of α1b-AR KO were significantly lower in response to D-amphetamine 2 mg/kg and 3 mg/kg (p<0.01 and p<0.05, respectively; Student's t-test).

For cocaine, amplitude of locomotor response was significantly altered by the genotype and the effect was independent of the dose of cocaine (genotype×dose: F_{2,46}=2.16 p>0.05, genotype: F_{1,46}=14.3, p<0.001; two-way Anova).

For morphine, amplitude of locomotor response was significantly altered by the genotype and the effect was independent of the dose of morphine (genotype×dose: F_{2,40}=1.22 p<0.05, genotype: F_{1,40}=6.9, p<0.05; two-way Anova). In the course of these experiments it was observed that, in KO mice, morphine induced a stereotyped walking behavior identical to that described following local perfusion of opiates in the nucleus accumbens. These responses, considered to be independent of the increased local release of DA (Kalivas et al., J. Pharmacol. Exp. Ther. 227:229-37, 1983), suggest the existence of at least two components in morphine-induced locomotor hyperactivity.

Effects of Prazosin on the Locomotor Responses of WT and α1b-AR KO Mice to D-Amphetamine, Cocaine and Morphine

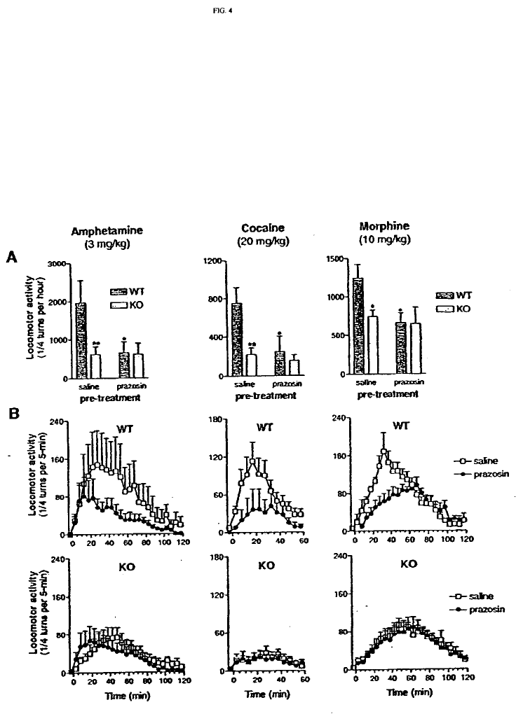

Since highest doses tested of D-amphetamine (3 mg/kg), cocaine (20 mg/kg) and morphine (10 mg/kg) significantly increased locomotor activity in KO mice, effects of prazosin (1 mg/kg) were measured in these conditions in WT and KO mice (FIG. 4). Prazosin significantly reduced the locomotor responses of WT mice to D-amphetamine (p<0.01, Student's t-test), cocaine (p<0.01, Student's t-test) and morphine (p<0.05, Student's t-test) but failed to modify the locomotor responses observed in α1b-AR KO mice. Moreover, prazosin pre-treatment abolished the locomotor differences between WT and α1b-AR KO mice (FIG. 4). This suggests that the inhibitory influence of prazosin observed in WT mice is solely due to the blockade of α1b-ARs and that locomotor differences between WT and KO mice are directly caused by the absence of α1b-AR in KO mice rather than to secondary neuro-developmental deficits.

Interestingly, following morphine administration, the stereotyped walking behavior previously described in KO mice was also observed in WT pre-treated with prazosin.

Locomotor Sensitization Induced by Repeated Administration of D-Amphetamine, Cocaine or Morphine in WT and α1b-AR KO Mice

For saline, locomotor responses decreased significantly with repeated injections in both WT (−5.3±2.1, F_{1,46}=6.4, p=0.015) and α1b-AR KO mice (−5.9±2.2, F_{1,45}=7.4, p=0.009). The rates of decrease were similar in both strains (F_{1,91}=0.039, p=0.84).

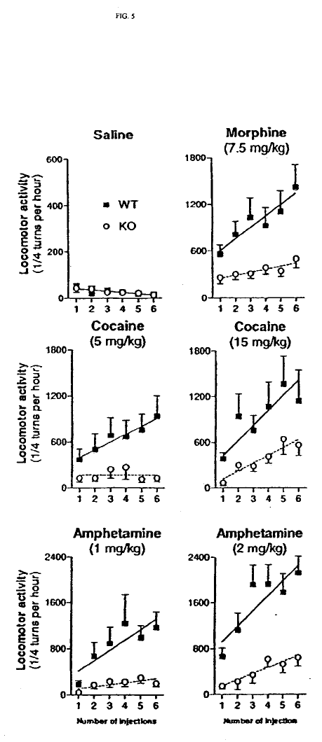

Repeated treatments with D-amphetamine (1-2 mg/kg), cocaine (5-15 mg/kg) or morphine (7.5 mg/kg) led to a progressive increase in the locomotor responses of WT animals that was correlated with the number of drug administrations. The rate of sensitization was evaluated by determining the slope of the “number of injections/response” curve (FIG. 5).

For morphine (7.5 mg/kg), locomotor response increased significantly with repeated injections in WT mice (146.7±51.4, F_{1,63}=8.11, p=0.006), but not in alb-AR KO mice (38.17±19.48, F_{1,81}=3.840, p=0.0535). The rate of sensitization was lower in α1b-AR KO than in WT mice (F_{1,44}=4.5, p=0.036).

For cocaine (5 mg/kg), locomotor response increased significantly with repeated injections in WT mice (slope: 101.0±47.4, F_{1,27}=4.52, p=0.042), but not in α1b-AR KO mice (−0.155±20.96, F_{1,40}<0.0001, p=0.99). The rate of sensitization was lower in α1b-AR KO than in WT mice (F_{1,67}=4.7, p=0.033). For cocaine (15 mg/kg), locomotor response increased significantly with repeated injections in both WT (198.2±48.19, F_{1,38}=16.92, p=0.0002) and α1b-AR KO mice (102.8±19.37, F_{1,47}=28.14, p<0.0001) and the rates of sensitization differed significantly between strains (F_{1,85}=3.96, p0.049).

For D-amphetamine (1 mg/kg), locomotor response increased significantly with repeated injections in WT mice (179.8±69.1, F_{1,33}=6.8, p=0.014). In α1b-AR KO mice, locomotor responses did not increase significantly with repeated injections (33.4±17.7, F_{1,34}=3.5, p=0.07). The rate of sensitization was lower in α1b-AR KO than in WT mice (F_{1,67}=4.3, p=0.042). For D-amphetamine (2 mg/kg), locomotor response increased significantly with repeated injections in both WT (265+71, F_{1,46}=13.69, p=0.0006) and α1b-AR KO mice (104.4±31, F_{1,46=10.98}, p=0.0018). However, the rate of sensitization was lower in α1b-AR KO than in WT mice (F_{1,92}=4.23, p=0.042).

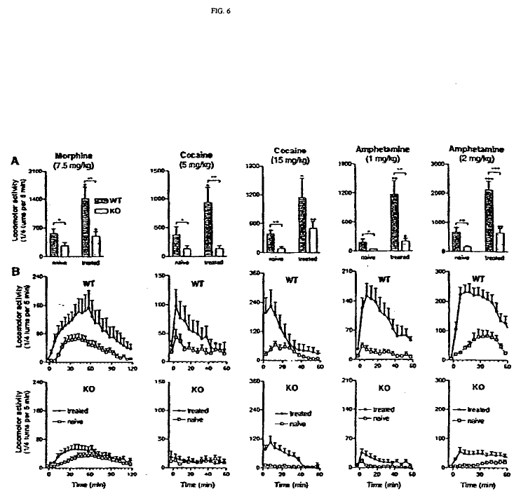

For both WT and α1b-AR KO mice, the locomotor responses of naive and drug-treated mice were compared (FIG. 6). In WT mice, locomotor responses of animals pretreated with drugs were higher than locomotor responses of naive animals, for all drugs tested. In KO mice, there was no significant difference in the locomotor responses of naive and drug-treated mice for cocaine (5 mg/kg). For other doses and other drugs tested, locomotor responses of drug-treated mice were higher than locomotor responses of naive animals. However, locomotor responses of α1-AR KO drug-treated mice were significantly lower than those of WT drug-treated mice.

Rewarding Properties of Cocaine and Morphine in WT and α1b-AR KO Mice

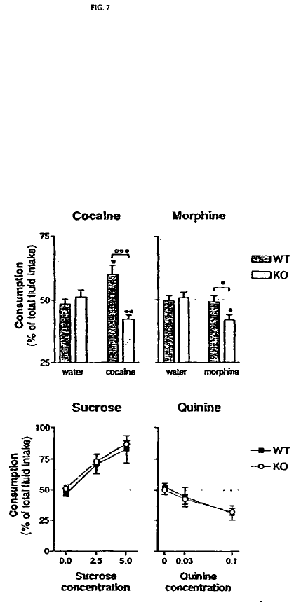

Rewarding properties of cocaine and morphine were assessed in a two-bottle choice paradigm adapted from the method described by Ferraro et al. (Ferraro et al., Pharmacol. Biochem. Behav. 66:397-401, 2000) for cocaine and Borg and Taylor (Borg et al., Biochem. Behav. 47:633-46, 1994) for morphine. In this test, WT and α1b-AR KO mice consumptions of cocaine and morphine were different. WT mice exhibited a preference for cocaine (p<0.05) but not for morphine, whereas KO mice displayed an aversion for both drugs (p<0.01 and p<0.05, respectively for cocaine and morphine) (FIG. 7, top).

No significant difference could be found between the two groups of animals for either sucrose preference (genotype: F_{1,64}=1.04, p=0.31 concentration×genotype: F_{2,64}=0.11, p=0.89 two-way RM Anova) or quinine aversion (genotype: F_{1,64}=0.4, p=0.53 concentration×genotype: F_{2,64}=0.28, p=0.75 two-way RM Anova) (FIG. 7, bottom), indicating that differences observed between genotypes were not related to differences in taste perception.

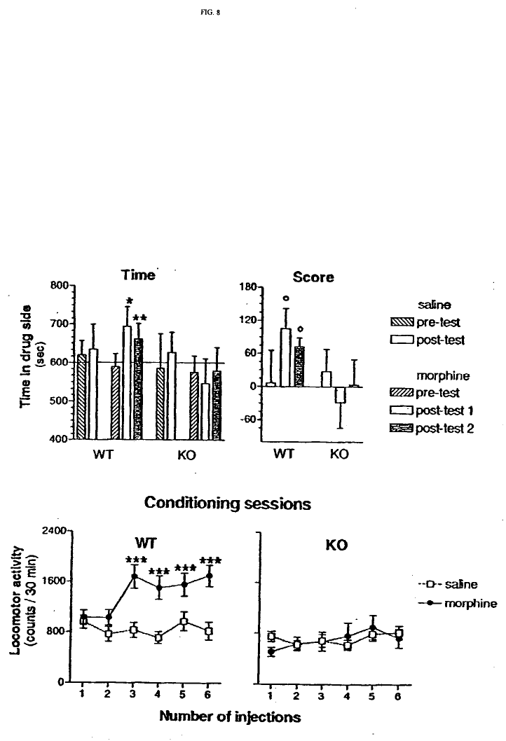

Since WT mice did not exhibit a clear preference for morphine in the oral consumption test, rewarding properties of morphine (5 mg/kg, s.c.) were also tested in the conditioned place preference (CPP) paradigm. A significant CPP was induced by morphine in WT (p<0.05) but not in KO mice (FIG. 8, top).

Locomotor activity was recorded during the conditioning sessions following either saline or morphine injection. In WT animals, differences between locomotor responses to saline and morphine were significantly influenced by the number of injections (treatment×number of injections: F_{5,50}=7.316, p<0.001, two-way RM Anova). In KO mice, no significant difference between locomotor responses to saline and morphine was observed (treatment: F_{1,69}=0.0155, p=0.903; treatment×number of injections: F_{5,60}=1.581, p=0.179). This indicates that, in these conditions, repeated morphine injections induce a behavioral sensitization in WT but not in KO mice (FIG. 8, bottom).

Effects of D-Amphetamine on Extracellular DA Levels in the Nucleus Accumbens and on Locomotor Activity of α1bAR-KO and WT Mice

Basal DA dialysate from the nucleus accumbens of α1bAR-KO mice was significantly lower (−28%) than that of WT (1.26±0.01 and 1.86±0.02 pg DA/20 μl, respectively; (F(_{1,119})=67.20, p<0.001, Two-way ANOVA). The localization of dialysis probes in the nucleus accumbens of mice and rat brains are shown in FIG. 9.

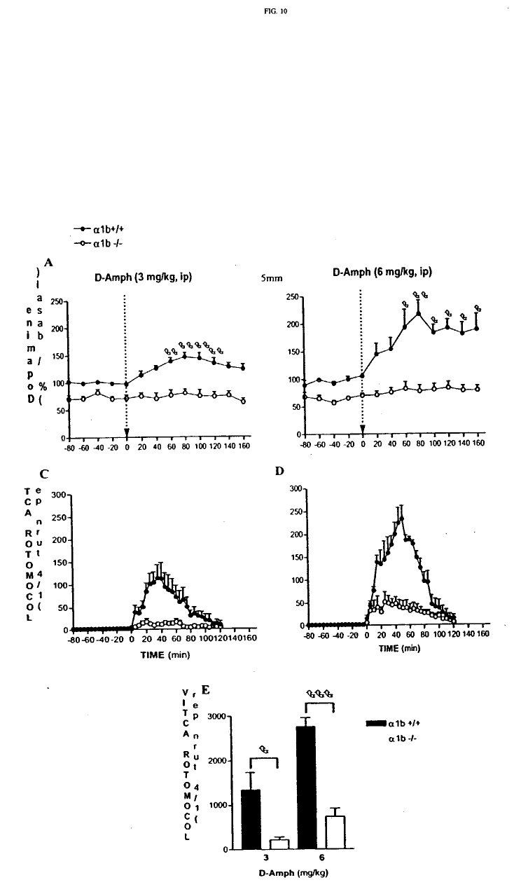

As expected, D-amphetamine (3 and 6 mg/kg, i.p.) enhanced extracellular DA levels in the nucleus accumbens of WT mice (F(_{1,80})=82.89, p<0.001 and F(_{1,37})=59.34, p<0.001, Two-way ANOVA, for 3 and 6 mg/kg, respectively) (FIGS. 10A and 10B). In α1bAR-KO mice, 3 mg/kg D-amphetamine did not modify basal extracellular DA levels (F(_{1,55})=0.655, p=0.421, Two-way ANOVA). Following 6 mg/kg D-amphetamine however, a slight mean increase (+25%) in α1bAR-KO extracellular DA levels was noticed (F(_{1,64})=7.1, p<0.01, Two-way ANOVA) although no individual point was significantly different from mean basal DA values (p>0.05. Dunnett's multiple comparison test) (FIGS. 10A and 10B).

Recording of locomotor activities indicated significant effects of D-amphetamine both in WT (F(_{1,135})=141.5, p<0.001 and F(_{1,16})=718.2, p<0.001, for 3 and 6 mg/kg D-amphetamine, respectively) and α1bAR-KO mice (F(_{1,135})=71.54, p<0.001 and F(_{1,136})=84.23, p<0.001 for 3 and 6 mg/kg D-amphetamine, respectively) (FIGS. 10C and 10D). However, locomotor hyperactivities of WT mice were significantly higher than those of α1bAR-KO mice (1352±389 vs 219±62, p<0.05, t(_{1,4})=2.876, Student's t-test with Welch's correction, and 2758±199 vs 734±184, p<0.001, t(_{1,8})=7.459, Student's t-test for 3 and 6 mg/kg D-amphetamine and for WT and α1bAR-KO mice, respectively) (FIG. 10E). Differences in D-amphetamine-induced increases in dialysate DA levels between α1bAR-KO and WT mice were not expected since, in rats, an α1-adrenergic antagonist, prazosin, inhibits D-amphetamine-induced locomotor hyperactivity without modifying extracellular DA responses in the nucleus accumbens (See Darracq et al., J. Neurosci. 18:2729-39, 1998).

Effects on DA Levels of the Local Perfusion of D-Amphetamine in the Nucleus Accumbens of C57BL6/J Mice and Sprague-Dawley Rats

Local perfusion of D-amphetamine in the nucleus accumbens was used to quantify non functional DA release. Experiments indicates that 3 μM D-amphetamine induces a DA release in WT mice more than 5-fold lower than previously found in rats (See Darracq et al., J. Neurosci. 18:2729-39, 1998). Because of the mixed genetic background of WT and α1bAR-KO mice, D-amphetamine dose-response curves were performed in C57BL6/J mice and compared in the same experimental conditions to those of Sprague-Dawley rats.

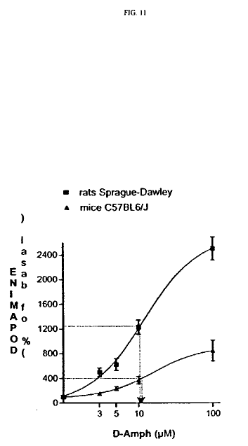

As found in rats, perfusion of D-amphetamine in mice nucleus accumbens up to 100 μM did not induce any locomotor hyperactivity. FIG. 11. indicates that DA release is concentration-dependent and at least three-fold lower in C57BL6/J mice than in Sprague-Dawley rats (F(_{4,185})=63.19, p<0.001 for D-amphetamine concentrations and F(_{1,185})=87.63, p<0.001 for comparison between rodent species). However, EC50's were found to be not significantly different (11.8±1.3 and 15.6±1.1 μM, for rats and mice, respectively; p>0.05, Student's t-test).

### Other Embodiments

It is to be understood that, while the invention has been described in conjunction with the detailed description thereof, the foregoing description is intended to illustrate and not limit the scope of the invention, which is defined by the scope of the appended claims. Other aspects, advantages, and modifications are within the scope of the following claims. amphetamine, respectively) and α1bAR-KO mice (F(_{1,135})=71.54, p<0.001 and F(_{1,136})=84.23, p<0.001 for 3 and 6 mg/kg D-amphetamine, respectively) (FIGS. 10C and 10D). However, locomotor hyperactivities of WT mice were significantly higher than those of α1bAR-KO mice (1352±389 vs 219±62, p<0.05, t(_{1,4})=2.876, Student's t-test with Welch's correction, and 2758±199 vs 734±184, p<0.001, t(_{1,8})=7.459, Student's t-test for 3 and 6 mg/kg D-amphetamine and for WT and α1bAR-KO mice, respectively) (FIG. 10E). Differences in D-amphetamine-induced increases in dialysate DA levels between α1bAR-KO and WT mice were not expected since, in rats, an α1-adrenergic antagonist, prazosin, inhibits D-amphetamine-induced locomotor hyperactivity without modifying extracellular DA responses in the nucleus accumbens (See Darracq et al., J. Neurosci. 18:2729-39, 1998).

Effects on DA Levels of the Local Perfusion of D-Amphetamine in the Nucleus Accumbens of C57BL6/J Mice and Sprague-Dawley Rats

Local perfusion of D-amphetamine in the nucleus accumbens was used to quantify non functional DA release. Experiments indicates that 3 μM D-amphetamine induces a DA release in WT mice more than 5-fold lower than previously found in rats (See Darracq et al., J. Neurosci. 18:2729-39, 1998). Because of the mixed genetic background of WT and α1bAR-KO mice, D-amphetamine dose-response curves were performed in C57BL6/J mice and compared in the same experimental conditions to those of Sprague-Dawley rats.

As found in rats, perfusion of D-amphetamine in mice nucleus accumbens up to 100 μM did not induce any locomotor hyperactivity. FIG. 11 indicates that DA release is concentration-dependent and at least three-fold lower in C57BL6/J mice than in Sprague-Dawley rats (F(_{4,185})=63.19, p<0.001 for D-amphetamine concentrations and F(_{1,185})=87.63, p<0.001 for comparison between rodent species). However, EC50's were found to be not significantly different (11.8±1.3 and 15.6±1.1 μM, for rats and mice, respectively; p>0.05, Student's t-test).

### Other Embodiments

It is to be understood that, while the invention has been described in conjunction with the detailed description thereof, the foregoing description is intended to illustrate and not limit the scope of the invention, which is defined by the scope of the appended claims. Other aspects, advantages, and modifications are within the scope of the following claims.

TABLE 1
Tissue contents of DOPAC, DA and norepinephrine in
wild type (WT) and αlb-AR knockout (KO) mice

| | | DOPAC | Dopamine | DOPAC/ | Norepinephrine |
|---|---|---|---|---|---|
| | | (μg/g of protein) | (μg/g of protein) | dopamine | (μg/g of protein) |
| Prefrontal | WT | 0.585_0.084 | 1.07_0.23 | 0.571_0.062 | 4.31_0.33 |
| Cortex | KO | 0.458_0.116 | 1.03_0.16 | 0.451_0.061 | 3.70_0.12 |
| Nucleus | WT | 18.3_2.2 | 113_12 | 0.160_0.009 | ND |
| Accumbens | KO | 15.4_2.5 | 95_14 | 0.159_0.008 | ND |
| Striatum | WT | 14.8_1.5 | 172_25 | 0.117_0.015 | ND |
| | KO | 13.4_0.9 | 195_25 | 0.090_0.009 | ND |

N = 4 animals per group.

I claim:

1. A method of identifying a modulator of alpha1b-adrenergic receptor activity, comprising the steps of:
(i) providing a first mammal, said first mammal containing a modified alpha1b-adrenergic receptor gene;
(ii) administering to said first mammal a candidate agent;
(iii) measuring a detectable phenotype of said first mammal;
(iv) comparing said detectable phenotype of said first mammal with a detectable phenotype of a second mammal;
wherein an alteration in the detectable phenotype of said first mammal compared to the second mammal indicates that said candidate agent is a modulator of alpha1b-adrenergic receptor activity.

2. The method of claim 1, wherein said modified alpha1b-adrenergic receptor gene is a non-functional alpha1b-adrenergic receptor gene.

3. The method of claim 1, wherein said modified alpha1b-adrenergic receptor gene is an alpha1b-adrenergic receptor gene having reduced function as compared to a wild-type alpha1b-adrenergic receptor gene.

4. The method of claim 1, wherein said detectable phenotype is selected from the group consisting of an increase in an alpha1b-adrenergic receptor activity, a decrease in an alpha1b-adrenergic receptor activity, a restoration of an alpha1b-adrenergic receptor activity such that the activity is similar to the activity of a wild-type alpha1b-adrenergic receptor, locomotor response, the level of extracellular dopamine in the nucleus accumbens, addiction to one or more addictive compounds, and conditioned place preference.

5. The method of claim 1, wherein said second mammal does not have a modified alpha1b-adrenergic receptor gene.

6. The method of claim 1, wherein said candidate agent is selected from the group consisting of: alpha1b-adrenergic receptor agonists, inverse agonists and antagonists.

7. The method of claim 1, wherein said candidate agent is an alpha1b-adrenergic receptor agonist, and wherein said alpha1b-adrenergic receptor activity is increased in the second mammal relative to the first mammal.

8. The method of claim 1, wherein said candidate agent is an alpha1b-adrenergic receptor antagonist, and wherein said alpha1b-adrenergic receptor activity is decreased in the second mammal relative to the first mammal.

9. The method of claim 1, wherein said administration is selected from the group consisting of oral administration and parenteral administration.

10. The method of claim 9, wherein said parenteral administration is selected from the group consisting of subcutaneous administration, subdermal administration, intraarterial administration, intravenous administration, intraperitoneal administration, topical administration, ophthalmic administration, nasal administration, and intramuscular administration.

11. A method of identifying a modulator of alpha1b-adrenergic receptor activity, comprising the steps of:
(i) administering to a test mammalian cell containing a modified alpha1b-adrenergic receptor gene a candidate agent;
(ii) measuring a detectable response by said test mammalian cell;
(iii) comparing said detectable response of said test mammalian cell with a reference response;
wherein a change in the detectable response of said test mammalian cell relative to said reference response indicates that said candidate agent is a modulator of alpha1b-adrenergic receptor activity.

12. The method of claim 11, wherein said modified alpha1b-adrenergic receptor gene is a non-functional alpha1b-adrenergic receptor gene.

13. The method of claim 11, wherein said modified alpha1b-adrenergic receptor gene is an alpha1b-adrenergic receptor gene having reduced function as compared to a wild-type alpha1b-adrenergic receptor gene.

14. The method of claim 11, wherein said detectable response is a change in the level of dopamine secreted by said test mammalian cell.

15. The method of claim 11, wherein said reference response is obtained from a mammalian cell that does not have a modified alpha1b-adrenergic receptor gene.

16. The method of claim 11, wherein said candidate agent is selected from the group consisting of alpha1b-adrenergic receptor agonists and antagonists.

17. The method of claim 11, wherein said candidate agent is an alpha1b-adrenergic receptor agonist, and wherein said alpha1b-adrenergic receptor activity is increased in the reference response relative to said test mammalian cell.

18. The method of claim 11, wherein said candidate agent is an alpha1b-adrenergic receptor antagonist, and wherein said alpha1b-adrenergic receptor activity is decreased in the reference response relative to said test mammalian cell.

19. The method of claim 11, wherein said test mammalian cell is obtained from a rodent.

20. The method of claim 19, wherein said rodent is a rat or a mouse.

21. A method of identifying a compound that inhibits or reduces drug addiction, comprising the steps of:
(i) providing a first mammal, said first mammal containing a modified alpha1b-adrenergic receptor gene, wherein said modified alpha1b-adrenergic receptor gene is selected from the group consisting of a non-functional alpha1b-adrenergic receptor gene and an alpha1b-adrenergic receptor gene with reduced function as compared to a wild-type alpha1b-adrenergic receptor gene;
(i) administering to said first mammal a candidate agent;
(ii) measuring a detectable behavior of said first mammal, wherein said detectable behavior is correlated with an addiction;
(iii) comparing said detectable behavior of said first mammal with a detectable behavior of a second mammal, wherein said second mammal does not have a modified alpha1b-adrenergic receptor gene;
wherein a decrease in the detectable behavior of said second mammal relative to said first mammal indicates that said candidate agent is a compound that inhibits or reduces addiction.

22. The method of claim 21, wherein said candidate agent is an antagonist or inverse agonist selective for the alpha1b-adrenergic receptor.

23. The method of claim 21, wherein said candidate agent is selected from the group consisting of alpha1b-adrenergic receptor agonists, inverse agonists, and antagonists.

24. The method of claim 21, wherein said administration is selected from the group consisting of oral administration and parenteral administration.

25. The method of claim 24, wherein said parenteral administration is selected from the group consisting of subcutaneous administration, subdermal administration, intraarterial administration, intravenous administration, intraperitoneal administration, topical administration, ophthalmic administration, nasal administration, and intramuscular administration.

26. A method of identifying a compound that promotes addiction, comprising the steps of:
(i) providing a first mammal, said first mammal containing a modified alpha1b-adrenergic receptor gene, wherein said modified alpha1b-adrenergic receptor gene is selected from the group consisting of a non-functional alpha1b-adrenergic receptor gene and an alpha1b-adrenergic receptor gene with reduced function as compared to a wild-type alpha1b-adrenergic receptor gene;
(ii) administering to said first mammal a candidate agent;
(iii) measuring a detectable behavior of said first mammal, wherein said detectable behavior is correlated with an addiction;
(iv) comparing said detectable behavior of said first mammal with a detectable behavior of a second mammal, wherein said second mammal does not have a modified alpha1b-adrenergic receptor gene;
wherein an increase in the detectable behavior of said second mammal relative to said first mammal indicates that said candidate agent is a compound that promotes addiction.

27. The method of claim 26, wherein said candidate agent is an agonist selective for the alpha1b-adrenergic receptor.

28. The method of claim 26, wherein said candidate agent is selected from the group consisting of: alpha1b-adrenergic receptor agonists, inverse agonists, and antagonists.

29. The method of claim 26, wherein said administration is selected from the group consisting of oral administration and parenteral administration.

30. The method of claim 29, wherein said parenteral administration is selected from the group consisting of subcutaneous administration, subdermal administration, intraarterial administration, intravenous administration, intraperitoneal administration, topical administration, ophthalmic administration, nasal administration, and intramuscular administration.

31. A method of identifying an inducer of an alpha1b-adrenergic receptor-associated disorder, comprising the steps of:
(i) providing a first mammal, said first mammal containing a modified alpha1b-adrenergic receptor gene;
(ii) administering to said first mammal a candidate agent;
(iii) measuring a detectable phenotype of said first mammal;
(iv) comparing said detectable behavior of said first mammal with a detectable behavior of a second mammal, wherein said second mammal does not have a modified alpha1b-adrenergic receptor gene;
wherein an increase in the detectable behavior of said second mammal relative to said first mammal indicates that said candidate agent is an inducer of an alpha1b-adrenergic receptor-associated disorder.

32. The method of claim 31, wherein said modified alpha1b-adrenergic receptor gene is a non-functional alpha1b-adrenergic receptor gene.

33. The method of claim 31, wherein said modified alpha1b-adrenergic receptor gene is an alpha1b-adrenergic receptor gene having reduced function as compared to a wild-type alpha1b-adrenergic receptor gene.

34. The method of claim 31, wherein said detectable behavior is selected from the group consisting of a change in locomotor response, addiction to one or more addictive compounds, and conditioned place preference.

35. The method of claim 31, wherein said second mammal does not have a modified alpha1b-adrenergic receptor gene.

36. The method of claim 31, wherein said candidate agent is an alpha1b-adrenergic receptor agonist, and wherein said alpha1b-adrenergic receptor activity is increased in the second mammal relative to the first mammal.

37. The method of claim 31, wherein said administration is selected from the group consisting of oral administration and parenteral administration.

38. The method of claim 37, wherein said parenteral administration is selected from the group consisting of subcutaneous administration, subdermal administration, intraarterial administration, intravenous administration, intraperitoneal administration, topical administration, ophthalmic administration, nasal administration, and intramuscular administration.

39. A method of identifying a repressor of an alpha1b-adrenergic receptor-associated disorder, comprising the steps of:
(i) providing a first mammal, said first mammal containing a modified alpha1b-adrenergic receptor gene;
(ii) administering to said first mammal a candidate agent;
(iii) measuring a detectable phenotype of said first mammal;
(iv) comparing said detectable behavior of said first mammal with a detectable behavior of a second mammal, wherein said second mammal does not have a modified alpha1b-adrenergic receptor gene;
wherein a decrease in the detectable behavior of said second mammal relative to said first mammal indicates that said candidate agent is a repressor of an alpha1b-adrenergic receptor-associated disorder.

40. The method of claim 39, wherein said modified alpha1b-adrenergic receptor gene is a non-functional alpha1b-adrenergic receptor gene.

41. The method of claim 39, wherein said modified alpha1b-adrenergic receptor gene is an alpha1b-adrenergic receptor gene having reduced function as compared to a wild-type alpha1b-adrenergic receptor gene.

42. The method of claim 39, wherein said detectable behavior is selected from the group consisting of a change in locomotor response, addiction to one or more addictive compounds, and conditioned place preference.

43. The method of claim 39, wherein said second mammal does not have a modified alpha1b-adrenergic receptor gene.

44. The method of claim 39, wherein said candidate agent is an alpha1b-adrenergic receptor antagonist, and wherein said alpha1b-adrenergic receptor activity is decreased in the second mammal relative to the first mammal.

45. The method of claim 39, wherein said administration is selected from the group consisting of oral administration and parenteral administration.

46. The method of claim 45, wherein said parenteral administration is selected from the group consisting of subcutaneous administration, subdermal administration, intraarterial administration, intravenous administration, intraperitoneal administration, topical administration, ophthalmic administration, nasal administration, and intramuscular administration.

47. The modulator identified by the method of claim 1.

48. The modulator identified by the method of claim 11.

49. The compound identified by the method of claim 21.

50. The compound identified by the method of claim 26.

51. The agonist identified by the method of claim 31.

52. The antagonist identified by the method of claim 39.
